(19) United States
(12) Patent Application Publication
(10) Pub. No.: US 2006/0063836 A1
(43) Pub. Date: Mar. 23, 2006

(54) Method for treating renal disease

(76) Inventors: Richard J. Roman, Brookfield, WI (US); Annette J. Dahly-Vernon, Germantown, WI (US); Mukut Sharma, Wauwatosa, WI (US)

Correspondence Address: QUARLES & BRADY LLP, 411 E. WISCONSIN AVENUE, SUITE 2040, MILWAUKEE, WI, 53202-4497, US

(21) Appl. No.: 11/229,241

(22) Filed: Sep. 16, 2005

Related U.S. Application Data
(60) Provisional application No. 60/610,465, filed on Sep. 16, 2004.

(51) Int. Cl. A61K 31/202

(52) U.S. Cl. 514/560

(57) ABSTRACT

A method for preventing and treating a renal disorder in a human or non-human animal is disclosed. The method involves administering 20-HETE or a 20-HETE agonist to the human or non-human animal in an amount sufficient to prevent or treat the renal disorder.

### CROSS-REFERENCE TO RELATED APPLICATIONS

This application claims the benefit of U.S. provisional application 60/610,465, filed on Sep. 18, 2004, which is incorporated by reference in its entirety.

### STATEMENT REGARDING FEDERALLY SPONSORED RESEARCH OR DEVELOPMENT

This invention was made with United States government support awarded by the following agency: NIH HL-36279. The United States has certain rights in this invention.

### BACKGROUND OF THE INVENTION

Diabetes and hypertension are the leading causes of end-stage renal disease (ESRD). Despite effective medications, compliance and drug costs are serious problems and only a small percentage of patients achieve adequate life-long control of blood pressure or their diabetes. As a result, the incidence of ESRD is increasing as the population ages and becomes more obese. The cost to the U.S. federal government for the treatment of ESRD exceeds 15 billion dollars a year.

Current treatment options for ESRD include kidney dialysis and transplant. In addition to the high cost associated with these two treatments, dialysis only provides filtration but not other functions of the kidney and kidney transplant has the problems of organ shortage and rejection.

Recently, TGF-β has been identified as a target for treating diabetes- and hypertension-induced nephropathies since TGF-β expression has been found to be upregulated in kidney in patients and animal models of these diseases (Noble N A and Border W A, Sem Nephrol 17:455-466, 1997; Reeves W B and Anderoli T E, Proc Natl Acad Sci 97:7667-7669, 2000; Sharma K and McGowan T. Cytokine Growth Factor Rev 11:115-123, 2000; Sharma K et al., Diabetes 46:854-859, 1997; Yamamoto T et al., Proc Nat'l Acad Sci 90:1814-1818, 1993; Yamamoto T et al., Kidney Int 49:461-469, 1996). Diabetes- and hypertension-induced nephropathies are characterized by the early development of proteinuria which accelerates the progression of renal disease by, for example, promoting the development of glomerular lesions (e.g., glomerulosclerosis), and TGF-β overexpression is believed to be a critical factor in this process (Dahly A J et al., Am J Physiol Regul Integr Comp Physiol 283:R757-767, 2002; Border W A et al. N Engl J Med 331:1286-1292, 1994; Sanders P W Hypertension 43:142-146, 2004; McCarthy E T et al., J Am Soc Nephrol 14:84A, 2003; Bottinger E P et al., J Am Soc Nephrol 10:2600-2610, 2002; August P et al., Kidney Int Suppl 87:S99-104, 2003; Ziyadeh F N et al., Proc Nat'l Acad Sci USA 97:8015-8020, 2000; 175, 202, 218, 265, 266). For example, TGF-β has been found to directly increase the permeability of isolated glomeruli to albumin (Sharma R et al., Kidney Int 58:131 -136, 2000), indicating a direct role of TGF-β in the induction of proteinuria. TGF-β has also been found to increase the production of extracellular matrix and promotes the development of glomerulosclerosis and renal interstitial fibrosis (Pavenstadt H et al., Physiol Rev 83:253-307, 2003; Border W A et al. N Engl J Med 331:1286-1292, 1994; and Sanders P W Hypertension 43:142-146, 2004). Importantly, blocking the activity of TGF-β by either TGF-β antibodies or antisense oligonucleotides has been shown to reduce the degree of proteinuria and glomerular damage (Dahly A J et al., Am J Physiol Regul Integr Comp Physiol 283:R757-767, 2002; Ziyadeh F N et al., Proc Nat'l Acad Sci USA 97:8015-8020, 2000; Chen s et al., Biochem Biophys Res Commun 300:16-22, 2003; and Han D C et al., Am J Physiol 278F628-F634, 2000).

Increased TGF-β expression in kidney is also associated with kidney transplantation rejection (Shihab F S et al. Kidney Int 50:1904-1913, 1996; Shihab F S et al., J Am Soc Nephrol 6:286-294, 1995), various forms of glomerulosclerosis (Yamamoto T et al., Kidney Int 49:461-469, 1996; Yoshioka K et al., Lab Invest 68:154-163, 1993), Heyman nephritis (Shankland S J et al., Kidney Int 50:116-124, 1996), remnant kidney (Lee L et al., J Clin Invest 96:953-964, 1995; Wu L L et al., Kidney Int 51:1553-1567, 1997), ureteral obstruction (Kaneto H et al., Kidney Int 44:313-321, 1993), kidney diseases caused by radiation and immunosuppressive and nephrotoxic drugs such as cyclosporine, puromycin, cisplatin, and heavy metals (Oikawa T et al., Kidney Int 51:164-172, 1997; Sharma V K et al., Kidney Int 49:1297-1303, 1996; Shihab F S et al. Kidney Int 49:1141-1151, 1996; Jones C L et al., Am J Path 141:1381-1396, 1992; Ma L J et al. Kidney Int 65:106-115, 2004), and every animal model of renal injury that has been examined (Noble N A and Border W A, Sem Nephrol 17:455-466, 1997). Blocking TGF-β activity by its antibodies provided beneficial effects in cyclosporine- and puromycin-induced nephropathies (Ling H et al., J Am Soc Nephrol 14:377-388, 2003; Ma L J et al. Kidney Int 65:106-115, 2004).

The mechanism by which TGF-# initiates the development of proteinuria and renal injury is not clear. Identifying downstream respondents of TGF-β in this regard will provide additional and novel targets for the treatment of renal diseases associated with elevations in the expression of TGF-B in the kidney.

### BRIEF SUMMARY OF THE INVENTION

The present invention provides a method for preventing or treating a renal disorder in a human or non-human animal by administering 20-hydroxyeicosatetraenoic acid (20-HETE) or an agonist thereof to the human or non-human animal in an amount sufficient to prevent or treat the renal disorder.

### BRIEF DESCRIPTION OF THE SEVERAL VIEWS OF THE DRAWINGS

FIG. 1 shows the expression of TGF-β1 in the kidney of Sprague Dawley (SD) and Dahl S rats (a genetic model of salt-sensitive hypertension and hypertension-induced renal disease) fed an LS and HS for 7 days. Renal homogenates isolated from SD (lanes 1-3), Dahl S rats fed an LS diet (lanes 4-7), and Dahl S rats fed an HS diet (8% NaCl) for 7 days (lanes 8-11). Each lane was loaded with a homogenate (30 μg protein/lane) isolated from different animals (n=3 to 4 per group). *Indicates a significant difference versus the values seen in Dahl S rats fed an LS diet. HS-7, HS diet for 7 days.

FIG. 2 shows the effect of an HS diet and the role of TGF-β on permeability to albumin (P_{alb}) in glomeruli isolated from SD rats and Dahl S rats fed an LS and HS diet for 7 days or in Dahl S rats fed an HS diet that were treated with a TGF-β Ab (1D11-7). The TGF-β Ab used effectively neutralizes all three isoforms of TGF-β. Glomeruli were preincubated with vehicle or 10 ng/ml of TGF-β1 for 15 minutes at 37° C. and P_{alb} was measured. Numbers in parentheses indicate the number of glomeruli and number of rats studied per group. *Indicates a significant difference versus the values seen in Dahl S rats fed an LS diet. † Indicates a significant difference from the corresponding control value. HS-4, HS for 4 days. HS-7, HS for 7 days.

FIG. 3 shows the effects TGF-β1 (10 ng/ml) on production of 20-HETE by isolated glomeruli. A representative LC/MS chromatogram is presented in Panel 4A. TGF-β1 inhibited the formation of 20-HETE peak with a m/z of 319 that elutes at a retention time of 16 minutes. Panel B presents a summary of the results obtained from 6 experiments. † Indicates a significant difference from the corresponding control value.

FIG. 4 shows the effects a 20-HETE agonist on the changes in P_{alb} produced by TGF-β1. Glomeruli were pre-incubated with vehicle or TGF-β1 (10 ng/ml) for 15 minutes at 37° C. and changes in P_{alb} were determined. Glomeruli were pretreated with a stable 20-HETE agonist, 20-hydroxyeicosa-5(Z), 14(Z)-dienoic acid (WIT003), for 15 minutes at 37° C. and the P_{alb} response to TGF-β1 (10 ng/ml) was redetermined. Numbers in parentheses indicate the number of glomeruli and number of rats studied per group. \ Indicates a significant difference from the corresponding control value.

### DETAILED DESCRIPTION OF THE INVENTION

It is disclosed here that upregulation in renal TGF-β increases permeability of the glomerular filtration barrier to albumin and other macromolecules through inhibiting the glomerular production of 20-HETE. As such increase in glomerular permeability to albumin leads to proteinuria and further to other glomerular injuries (e.g., glomerulosclerosis and renal interstitial fibrosis), the present invention provides new tools for preventing and treating TGF β-related renal disorders as well as physical and pathological manifestations thereof.

In one aspect, the present invention relates to a method for preventing or treating a TGF β-related renal disorder in a human or non-human animal. The method involves administering 20-HETE or a 20-HETE agonist to the human or non-human animal in an amount sufficient to prevent or treat the renal disorder. By TGF β-related renal disorder, we mean a renal disease and physical and pathological manifestations thereof in which TGF-β expression is upregulated. Examples of such disorders include but are not limited to proteinuria, nephropathies induced by diabetes and hypertension (e.g., salt sensitive hypertension), kidney transplantation rejection, Heyman nephritis, remnant kidney nephropathy, ureteral obstruction nephropathy, and kidney diseases caused by radiation and immunosuppressive and nephrotoxic drugs such as cyclosporine, puromycin, cisplatin, and heavy metals. In one embodiment, the method of the present invention is employed to prevent or treat proteinuria or a proteinuria-related renal disorder. By proteinuria-related renal disorder, we mean a renal disease in which proteinuria is detected. In another embodiment, the method of the present invention is employed to prevent or treat diabetes- or hypertension-induced nephropathy.

Examples of 20-HETE agonists that can be used in the present invention include but are not limited to those disclosed in U.S. Pat. No. 6,395,781; Yu M et al., Eur J Pharmacol. 486:297-306, 2004; Yu M et al., Bioorg Med Chem. 11:2803-2821, 2003; and Alonso-Galicia M et al., Am J Physiol. 277:F790-796, 1999, all of which are herein incorporated by reference in its entirety. For example, 20-HETE agonists defined by the following formula as provided in U.S. Pat. No. 6,395,781 can be used in the present invention: 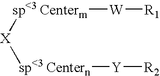
wherein R₁ is selected from the group consisting of carboxylic acid, phenol, amide, imide, sulfonamide, sulfonamide, active methylene, 1,3-dicarbonyl, alcohol, thiol; amine, tetrazole and other heteroaryl groups;
R₂ is selected from the group consisting of carboxylic acid, phenol, amide, imide, sulfonamide, sulfonamide, active methylene, 1,3-dicarbonyl, alcohol, thiol, amine, tetrazole and other heteroaryl;
W is a carbon chain (C₁ through C₂₅) and may be linear, cyclic, or branched and may comprise heteroatoms;
Y is a carbon chain (C₁ through C₂₅) and may be linear, cyclic, or branched and may comprise heteroatoms;
sp^{<3} Center is selected from the group consisting of vinyl, aryl, heteroaryl, cyclopropyl, and acetylenic moieties;
X is an alkyl chain that may be linear, branched, cyclic or polycyclic and may comprise heteroatoms;
m is 0, 1, 2, 3, 4 or 5; and
n is 0, 1, 2, 3, 4 or 5.

Preferably, a 20-HETE agonist defined by the above formula has a carboxyl or other ionizable group at either R₁ or R₂ and contains a double bond or other functional group at a distance equal to 14-15 carbons from the ionizable group (U.S. Pat. No. 6,395,781). More preferably, the 20-HETE agonist contains a length of 20-21 carbons, has a carboxyl or other ionizable group at either R₁ or R₂, contains a double bond or other functional group at a distance equal to 14-15 carbons from the ionizable group, and contains a hydroxyl group on the 20 or 21 carbon at either R₁ or R₂ (U.S. Pat. No. 6,395,781).

In one form, the present invention contemplates the use of one or more of the following 20-HETE agonists: 20-hydroxyeicosanoic acid, 20-hydroxyeicosa-5(Z),14(Z)-dienoic acid (WIT003), and N-methylsulfonyl-20-hydroxyeicosa-5(Z),14(Z)-dienamide.

The present invention is not limited by a specific route of administration. Suitable routes of administration for 20-HETE or a 20-HETE agonist include but are not limited to oral administration, intravenous administration, subcutaneous administration, intramuscular administration, and direct delivery into the kidney. Optimal dosages of 20-HETE or a particular 20-HETE agonist for preventing or treating a particular renal disorder via a particular route of administration can be readily determined by a skilled artisan.

The invention will be more fully understood upon consideration of the following non-limiting example.

### EXAMPLE

### 20-HETE Agonist Opposes TGF β-Induced Glomerular Injury

This example shows that transforming growth factor-beta (TGF-β) alters the glomerular permeability by inhibiting the glomerular production of 20-hydroxyeicosatetraenoic acid (20-HETE). Renal expression of TGF-β doubled in Dahl salt-sensitive (Dahl S) rats fed a high salt diet for 7 days and this was associated with a marked rise in permeability to albumin (P_{alb}) from 0.19±0.04 to 0.75±0.01 along with changes in the ultrastructure of the glomerular filtration barrier. Chronic treatment of Dahl S rats with a TGF-β neutralizing antibody prevented the increase in P_{alb} and preserved the structure of glomerular capillaries proving that hypertension-induced renal disease is dependent on increased formation and action of TGF-β. It had no effect on the rise in blood pressure produced by the high-salt diet. Preincubation of glomeruli isolated from Sprague Dawley (SD) rats with TGF-β1 (10 ng/ml) for 15 minutes increased P_{alb} from 0.01±0.01 to 0.60±0.02. This was associated with inhibition of the glomerular production of 20-HETE from 221±11 to 3.4±0.5 μg/30 min/mg protein. Pretreatment of SD glomeruli with a stable analog of 20-HETE, 20-hydroxyeicosa-5(Z), 14(Z)-dienoic acid, reduced baseline P_{alb} and opposed the effects of TGF-β to increase P_{alb}.

Materials and Methods

Dahl salt-sensitive rat model: Dahl salt-sensitive (S) rats exhibit many traits associated with salt-sensitive hypertension in humans (Campese V M. Hypertension 78:531-550, 1994; and Grimm C E et al., Hypertension 15:803-809, 1990). They are salt-sensitive (Iwai, J. Hypertension 9:I18-120, 1987; and Rapp J. P. Hypertension 4:753-763, 1982), insulin-resistant (Reft, G M et al., Hypertension 18:630-635, 1991) and hyperlipidemic (Raji, L et al., Kidney Int. 41:801-806, 1984; and O'Donnell, M P et al., Hypertension 20:651-658, 1992), and they rapidly develop proteinuria and glomerulosclerosis when challenged with a high salt (HS) diet (O'Donnell, M P et al., Hypertension 20:651-658, 1992; Roman R J et al., Hypertension 12:177-183, 1988; Roman R J et al., Hypertension 21:985-988, 1988; Roman R J et al., Am J Hypertens 10:63S-67S, 1997; and Tolins J P et al., Hypertension 16:452-461, 1990). The glomerular lesions that develop resemble those seen in patients with hypertension- and diabetes-induced nephropathy (McClellan W et al., Am J Kidney Dis 12:285-290, 1987; Ronstand G S et al., N Engl J Med 306:1276-1279, 1982; and Tierney W M et al., Am J Kidney Dis 13:485-493, 1989). The renal expression of transforming growth factor-β (TGF-β) is elevated in Dahl S rats fed a high salt (HS) diet and that chronic treatment of Dahl S rats with a TGF-β neutralizing antibody (Ab) for three weeks reduces proteinuria and the degree of glomerulosclerosis and fibrosis (Dahly A J et al., Am J Physiol Regul Integr Comp Physiol 283:R757-767, 2002).

General: Experiments were performed on 7-week-old Sprague Dawley (Taconic Labs) rats fed a normal-salt diet containing 1% NaCl (#5010, Purina) and Dahl salt-sensitive/John Rapp rats obtained from our colony maintained at the Medical College of Wisconsin. Rats were fed a purified diet (AIN76) purchased from Dytes, Inc. that contained either 0.4% (low salt, LS) or 8.0% NaCl (high salt, HS). To assess the role of TGF-β in altering proteinuria and P_{alb} during hypertension development, a group of the Dahl S rats fed a HS diet were treated with an intraperitoneal injection of a murine anti-TGF-β monoclonal Ab (0.5 mg/kg; 1D11; Genzyme Corp) or a control murine monoclonal Ab (13C4; antiverotoxin) every other day (Dasch J R et al., J Immunol 10:2109-2119, 1989). At the end of the treatment period, rats were placed overnight in metabolic cages for measurement of protein and albumin excretion (Dahly A J et al., Am J Physiol Regul Integr Comp Physiol 283 :R757-767, 2002). They were then anesthetized with halothane, and the kidneys were collected for measurement of the expression of TGF-β protein levels by Western blot (Hoagland K M et al., Hypertension 43:860-865, 2004) and for glomerular isolation for the measurement of P_{alb} and the production of 20-HETE. Catheters connected to radiotelemetry transmitters Data Science Inc.) were implanted into the femoral artery of 10 additional control and 10 1D11-treated Dahl S rats to determine the effects of anti-TGF-β therapy on the development of hypertension. Mean arterial pressure (MAP) was measured for 3 hours per day, between 9 AM and 12 PM, during a control period when rats were fed an LS diet and after they were fed an HS diet for 7 days.

Measurement of albumin Permeability (P_{alb}): Glomeruli were isolated using the sieving method described in Sharma R et al. (Kidney Int 58:131-136, 2000) and Savin V J et al. (J Am Soc Nephrol 3:1260-1269, 1992) in a media containing 5 g/dl of bovine serum albumin (BSA). In each experimental condition, P_{alb} was determined from the change in glomerular volume (ΔV) after exchange of the bath with medium containing 1 g/dL albumin. P_{alb} was calculated as 1-(ΔVₑₓₚₑᵣᵢₘₑₙₜₐₗ/ΔV_{control}), where glomeruli from Sprague Dawley rats fed a normal-salt diet were used to provide the control value for each experiment. To verify that lack of ΔVs in Dahl S rats were related to changes in P_{alb} rather than to changes in mechanical properties of glomeruli, additional studies were performed in which the glomeruli were exposed to a 5% solution of high molecular weight dextran. A change in the size of Dahl S glomeruli under these conditions_indicates that the lack of response to 1% albumin was attributable to an increase in P_{alb} (Savin V J et al., J Am Soc Nephrol 3:1260-1269, 1992).

In other experiments, we examined the interaction of TGF-β and 20-HETE on P_{alb} in glomeruli isolated from Sprague Dawley rats and Dahl S rats fed either an LS diet or an HS diet for 4 days. Glomeruli were preincubated with vehicle or TGF-β1 (10 ng/ml) for 15 minutes at 37° C. and changes in P_{alb} were determined. Glomeruli were also pretreated with a stable 20-HETE agonist, 20-hydroxyeicosa-5(Z), 14(Z)-dienoic acid (WIT003; 1 μmol/L; Taisho Pharmaceutical) (Alonso-Galicia, M et al., Am. J. Physiol. 277:F790-F796, 1999; and Yu, M et al., Bioorg. Med. Chem. 11:2803-2821, 2003), for 15 minutes at 37° C. and the P_{alb} response to TGF-β1 (10 ng/ml) was redetermined. A minimum of 5 glomeruli from each rat was studied, and these experiments were performed using ≧5 rats per treatment group.

Electron microscopy: Kidneys from Dahl S rats fed an LS diet and Dahl S rats fed an HS diet for 1 week and treated with 1D11 or vehicle were collected and fixed in a 4% glutaldehyde solution. Thin epon sections were prepared, stained with uranyl acetate and lead citrate, and examined at 16,000× using a transmission electron microscope (Hitachi H600).

Western blots: Homogenates were prepared from the kidneys of control Sprague Dawley rats and Dahl S rats fed an LS or HS diet for 7 days. Aliquots of the homogenates (30 μg protein) were separated on a 12.5% sodium dodecyl sulfate gel, transferred to a nitrocellulose membrane incubated with a primary TGF-β1 Ab (SC:146; Santa Cruz Biotechnology), followed by a secondary Ab (SC:2004; Santa Cruz Biotechnology) and developed using enhanced chemiluminescence as described in Hoagland K M et al., Hypertension 43:860-865, 2004. Membranes were poststained with Commassie blue to normalize results for potential differences in sample loading.

Liquid Chromatography/Mass Spectroscopy measurement of glomerular 20-HETE production: Glomeruli (approximately 20 μg protein) were incubated in a 0.1 mol/L KPO₄ buffer containing 1 mmol/L NADPH for 30 min at 37° C. in the presence and absence of TGF-β1 (10 ng/ml). Incubations were stopped by acidification with formic acid, homogenized, and the homogenate extracted with chloroform:methanol (2:1) after addition of 10 ng of internal standard, 14,15-epoxyeicosa-5(Z)-enoic-methyl sulfonylimide (EEZE). Samples were reconstituted in 50% acetonitrile, cleaned up using on online reverse-phase high performance liquid chromatography (HPLC) trapping column, and then the HETEs and epoxyeicosatrienoic acids (EETs) in the samples were separated using an isocratic step gradient on an 18C-RP 2×250 mm microbore HPLC (BetaBasic18 150×21 3 μm, Thermo.Hypersil-Keystone) using a mobile phase consisting of acetonitrile:water:acetic acid (57:43:0.1) for 20 minutes to resolve the HETEs followed by acetonitrile:water:acetic acid (63:37:0.1) for 15 minutes to resolve the EETs. Samples were ionized using negative ion electrospray and the peaks eluting with a mass/charge ratio (m/z) of 319 (HETEs and EETs) or 323 (internal standard) were isolated and monitored in the selective ion mass spectroscopy (MS) mode using an Agilent LSD ion trap mass spectrometer (Agilent Technologies 1100). The ratio of ion abundances in the peaks of interest (HETEs and EETs, m/z 319) versus that corresponding to the closely eluting internal standard (EEZE, m/z 323) were determined and compared with a standard curve generated over a range from 0.1 to 2 ng of 20-HETE and EETs with each batch of samples.

Statistics: Mean values±1 SE are presented. Significance of differences between mean values was determined using an ANOVA followed by the Student-Newman-Keuls post hoc test. A P<0.05 was considered significant.

Results

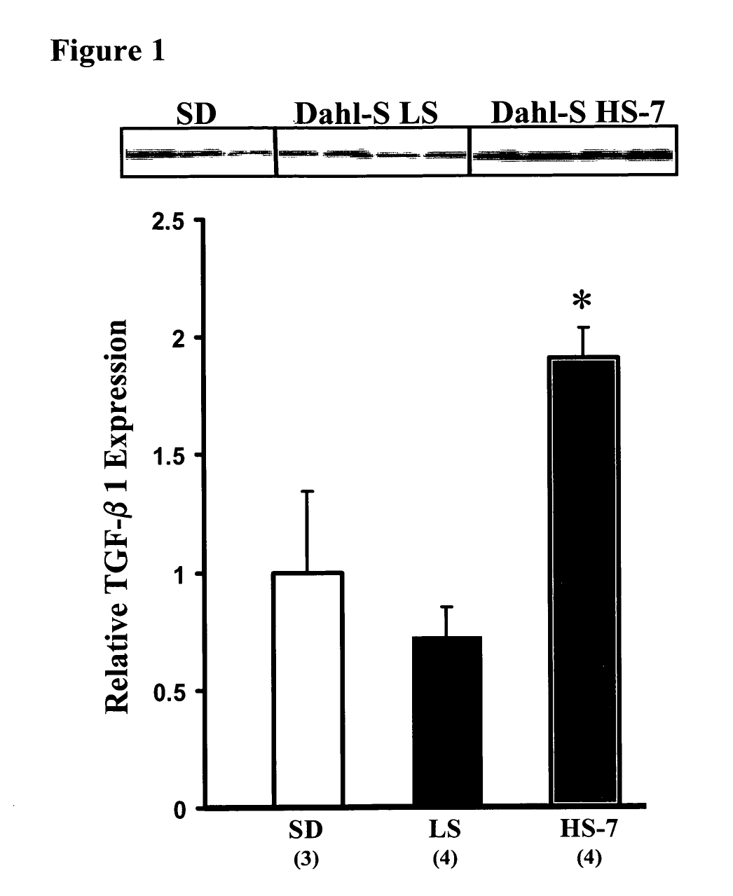

Effects of high salt diet on the renal expression of TGF-β1: The results of these experiments are presented in FIG. 1. The expression of TGF-β1 in the kidney more than doubled in Dahl S rats fed an HS diet for 1 week compared with the levels seen in Dahl S rats fed an LS diet.

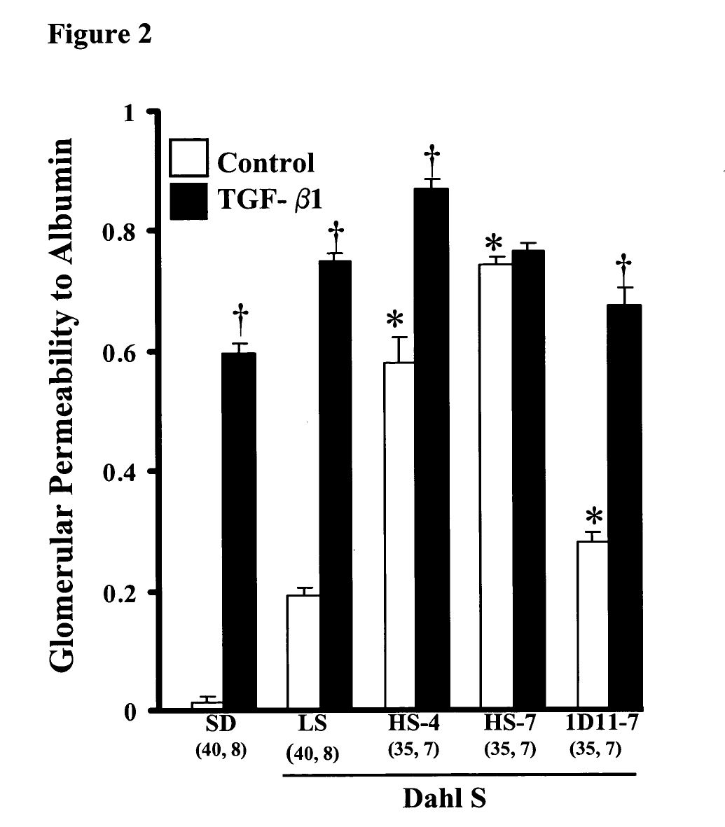

Effects of high salt diet on P_{alb}: A comparison of P_{alb} in Sprague Dawley and Dahl S rats fed an LS and HS diet at various times for up to a week are presented in FIG. 2. Baseline P_{alb} was significantly higher in Dahl S rats maintained on an LS diet than in control Sprague Dawley rats. P_{alb} increased in Dahl S rats fed an HS diet after only 4 days, and it reached a peak after 7 days. The increase in P_{alb} in Dahl S rats fed an HS for one week was associated with a significant rise in blood pressure from 121±2 to 136±3 mm Hg (n=10) and a marked increase in the excretion of protein from 47±8 mg/day to 217±31 mg/day (n=14). Similarly, albumin excretion rose from 27±9 mg/day to 129±26 mg/day, after Dahl S rats were fed an HS diet for 7 days.

Role of TGF-β in altering P_{alb} in Dahl S rats: A comparison of the effects of exogenous administration of TGF-β1 (10 ng/mL) on P_{alb} in glomeruli isolated from Sprague Dawley and Dahl S rats is also summarized in FIG. 2. TGF-β1 increased P_{alb} from 0.01±0.01 to 0.56±0.02 in glomeruli isolated from Sprague Dawley rats and from 0.19±0.01 to 0.75±0.01 in glomeruli isolated from Dahl S rats fed an LS diet. TGF-β1 also increased in P_{alb} in Dahl S rats fed an HS diet for 4 days, but it had no effect on P_{alb} in Dahl S rats fed an HS diet for 7 days, because the baseline P_{alb} in these rats was already near maximal.

Chronic treatment of Dahl S rats fed an HS diet with a TGF-β neutralizing Ab prevented the increase in baseline P_{alb}. Administration of TGFβ1 to these glomeruli still increased P_{alb}, similar to that seen in glomeruli isolated from control Sprague Dawley rats and Dahl S rats fed an LS diet. TGF-β1 Ab therapy had no effect on the rise in blood pressure. Blood pressure rose from 123±4 to 136±3 mm Hg (n=10) in Dahl S rats fed an HS diet that were treated with 1D11 for 7 days.

Electron microscopy: Electron micrographs of the ultrastructure of glomerular capillaries in Dahl S rats fed an LS or HS diet, and in those treated with the TGF-β Ab for 1 week, were obtained. The Dahl S rats fed an LS diet exhibited a normal appearance of the glomerular ultrafiltration barrier. In Dahl S rats fed an HS diet for 7 days, there was a retraction and fusion of the foot processes of podocytes and exposure of portions of the basement membrane. There was also swelling of the endothelial cells lining the glomerular capillaries, which changed their shape from a flattened to a more cubodial endothelium. These changes in the ultrastructure of glomerular filtration barrier in Dahl S rats fed an HS diet were prevented by administration of the TGFβ Ab.

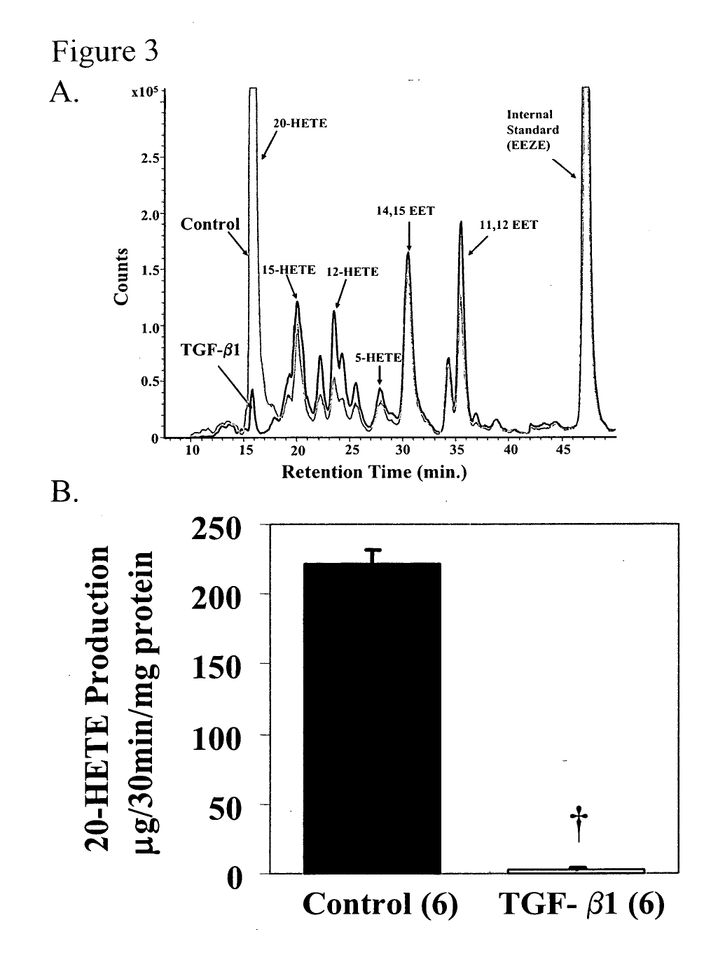

Effect of TGF-β on the glomerular production of 20-HETE: The effects of TGF-β on the production and metabolism of arachidonic acid (AA) by isolated glomeruli are presented in FIG. 3. Glomeruli incubated with AA produced a number of large peaks with an m/z of 319 that coelutes with 20-HETE, 15-HETE, 12-HETE, 5-HETE and 14,15-EET, 11,12-EET, 8,9-EET, and 5,6-EET standards (FIG. 3A). We further verified that the largest peak that elutes at 16 minutes after fragmentation produces an MS/MS spectrum with prominent secondary ions at m/z of 301, 273, 257, and 245, identical to that seen with a 20-HETE standard. Pretreatment of glomeruli with TGF-β1 selectively reduced the formation of 20-HETE by 97% (FIG. 3B) without affecting the formation of 15-, 12- or 5-HETE or EETs (FIG. 3A).

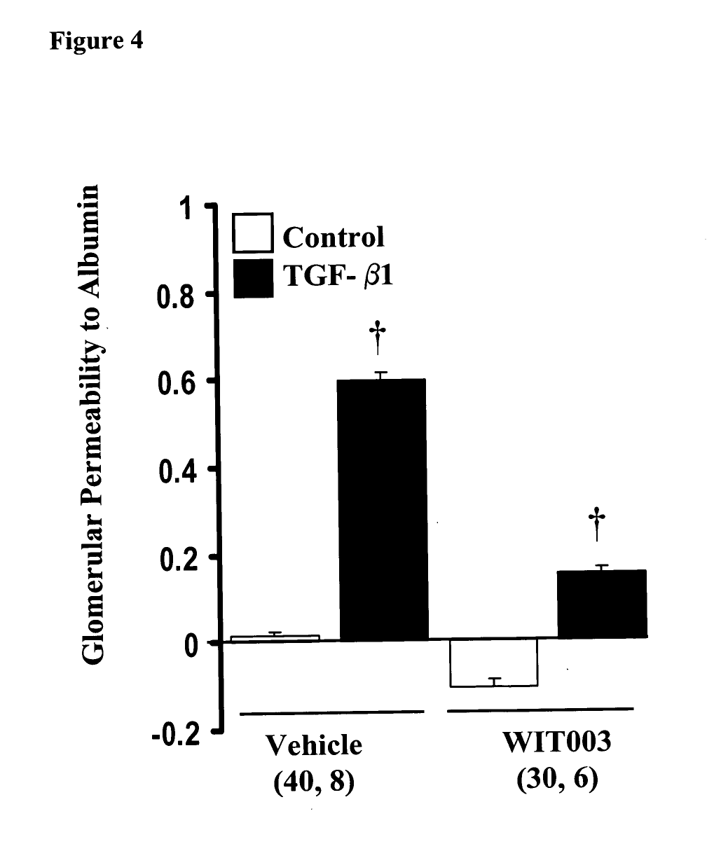

Effects of a 20-HETE agonist on P_{alb}: The effect of addition of a 20-HETE agonist on the changes in P_{alb} produced by TGF-β1 is summarized in FIG. 4. Pretreatment of glomeruli with a 20-HETE agonist reduced baseline P_{alb} and greatly attenuated the increase in P_{alb} produced by TGF-β1. Similar results were obtained with Dahl S rats maintained on an LS diet or fed an HS diet for 4 days. For example, TGF-β1 increased P_{alb} from 0.58±0.04 (n=25 glomeruli; 5 rats) to 0.87±0.02 (n=25; 5 rats) in glomeruli isolated from Dahl S rats fed an HS diet for 4 days. After pretreatment of glomeruli with the 20-HETE agonist, TGF-β1 P_{alb} only increased from 0.25±0.01 (n−25; 5 rats) to 0.40±0.01 (n=25; 5 rats).

The present invention is not intended to be limited to the foregoing example, but encompasses all such modifications and variations as come within the scope of the appended claims.

We claim:

1. A method for preventing or treating a renal disorder in a human or non-human animal comprising the step of:
administering an agent selected from the group consisting of 20-HETE and a 20-HETE agonist to the human or non-human animal in an amount sufficient to prevent to treat the renal disorder.

2. The method of claim 1, wherein the method is for preventing or treating a renal disorder in a human subject.

3. The method of claim 1, wherein the renal disorder is selected from the group consisting of proteinuria, diabetes-induced nephropathy, hypertension-induced nephropathy, kidney transplantation rejection, Heyman nephritis, remnant kidney nephropathy, ureteral obstruction nephropathy, and a kidney disease caused by radiation, a kidney disease caused by an immunosuppressive drug, and a kidney disease caused by a nephrotoxic drugs.

4. The method of claim 1, wherein the renal disorder is proteinuria.

5. The method of claim 1, wherein the renal disorder is diabetes-induced nephropathy

6. The method of claim 1, wherein the renal disorder is hypertension-induced nephropathy.

7. The method of claim 6, wherein the hypertension-induced nephropathy is salt sensitive hypertension-induced nephropathy.

8. The method of claim 1, wherein the agent is a 20-HETE agonist.

9. The method of claim 8, wherein the 20-HETE agonist is defined by the formula: 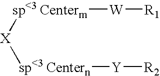
wherein R₁ is selected from the group consisting of carboxylic acid, phenol, amide, imide, sulfonamide, sulfonamide, active methylene, 1,3-dicarbonyl, alcohol, thiol, amine, tetrazole, and other heteroaryl groups;
R₂ is selected from the group consisting of carboxylic acid, phenol, amide, imide, sulfonamide, sulfonamide, active methylene, 1,3-dicarbonyl, alcohol, thiol, amine, tetrazole, and other heteroaryl;
W is a carbon chain (C₁ through C₂₅) and may be linear, cyclic, or branched and may comprise heteroatoms;
Y is a carbon chain (C₁ through C₂₅) and may be linear, cyclic, or branched and may comprise heteroatoms;
sp^{<3} Center is selected from the group consisting of vinyl, aryl, heteroaryl, cyclopropyl, and acetylenic moieties;
X is an alkyl chain that may be linear, branched, cyclic or polycyclic and may comprise heteroatoms;
m is 0, 1, 2, 3, 4 or 5; and
n is 0, 1, 2, 3, 4 or 5.

10. The method of claim 9 wherein the compound has a carboxyl or other ionizable group at either R₂ or R₂ and wherein the compound comprises a double bond or other functional group at a distance equal to 14-15 carbons from the ionizable group.

11. The method of claim 10, wherein the compound comprises a length of 20-21 carbons, has a carboxyl or other ionizable group at either R₁ or R₂, comprises a double bond or other functional group at a distance equal to 14-15 carbons from the ionizable group, and comprises a hydroxyl group on the 20 or 21 carbon at either R₁ or R₂.

12. The method of claim 8, wherein the 20-HETE agonist is selected from the group consisting of 20-hydroxyeicosanoic acid, 20-hydroxyeicosa-5(Z),14(Z)-dienoic acid (WIT003), and N-methylsulfonyl-20-hydroxyeicosa-5(Z), 14(Z)-dienamide.

13. The method of claim 12, wherein the 20-HETE agonist is 20-hydroxyeicosa-5(Z),14(Z)-dienoic acid (WIT003).

14. The method of claim 1 further comprising the step of:
observing improvement in kidney function in the human or non-human animal.

15. The method of claim 14, wherein the improvement is an improvement in proteinuria.
